(19) United States
(12) Patent Application Publication
(10) Pub. No.: US 2026/0248372 A1
(43) Pub. Date: Aug. 27, 2026

(54) DEFLECTABLE MEDICAL DEVICES

(71) Applicant: Boston Scientific Scimed, Inc., Maple Grove, MN (US)

(72) Inventors: Samuel Shreve, Minneapolis, MN (US); Matthew Paul Joseph DiCicco, Toronto (CA); Nicholas Lee Tassoni, Andover, MN (US)

(73) Assignee: Boston Scientific Scimed, Inc., Maple Grove, MN (US)

(21) Appl. No.: 19/541,818

(22) Filed: Feb. 17, 2026

Related U.S. Application Data
(60) Provisional application No. 63/759,743, filed on Feb. 18, 2025.

(51) Int. Cl. A61B 1/005; A61B 17/3207; A61B 18/14; A61B 18/00

(52) U.S. Cl. CPC A61B 1/0052; A61B 1/0055; A61B 1/0057; A61B 17/3207; A61B 18/1492; A61B 2018/00577; A61B 2018/00601

(57) ABSTRACT

Deflectable medical devices as well as methods for making and using deflectable medical devices are disclosed. An example deflectable medical device may include an elongate shaft having a distal end region. The distal end region may define a first radius of curvature when bending the distal end region in a first direction prior to having a pre-shaped curve formed therein. The distal end region may define a second radius of curvature when bending the distal end region in the first direction after having the pre-shaped curve formed therein. The distal end region may define a third radius of curvature when bending the distal end region in a second direction different from the first direction after having the pre-shaped curve formed therein. The second radius of curvature may be larger than the third radius of curvature.

### CROSS REFERENCE TO RELATED APPLICATIONS

This application claims the benefit of U.S. Provisional Patent Application Serial No. 63/759,743 filed on Feb. 18, 2025, the disclosure of which is incorporated herein by reference.

### TECHNICAL FIELD

The present disclosure pertains to medical devices, and methods for manufacturing medical devices. More particularly, the present disclosure pertains to elongated medical devices including a tubular member connected with other structures, and methods for manufacturing and using such devices

### BACKGROUND

A wide variety of medical devices have been developed for medical use, and more specifically for intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A deflectable medical device is disclosed. The deflectable medical device comprises: an elongate shaft having a distal end region; wherein the distal end region defines a first radius of curvature when bending the distal end region in a first direction prior to having a pre-shaped curve formed therein; wherein the distal end region defines a second radius of curvature when bending the distal end region in the first direction after having the pre-shaped curve formed therein; wherein the distal end region defines a third radius of curvature when bending the distal end region in a second direction different from the first direction after having the pre-shaped curve formed therein; and wherein the second radius of curvature is larger than the third radius of curvature.

Alternatively or additionally to any of the embodiments above, the first radius of curvature is smaller than the second radius of curvature.

Alternatively or additionally to any of the embodiments above, the first radius of curvature is larger than the second radius of curvature.

Alternatively or additionally to any of the embodiments above, the first radius of curvature is substantially equal to the second radius of curvature.

Alternatively or additionally to any of the embodiments above, the distal end region defines a first reach distance when bending the distal end region in the first direction prior to having the pre-shaped curve formed therein, wherein the distal end region defines a second reach distance when bending the distal end region in the first direction after having the pre-shaped curve formed therein, and wherein the first reach distance is different from the second reach distance.

Alternatively or additionally to any of the embodiments above, the second reach distance is larger than the first reach distance.

Alternatively or additionally to any of the embodiments above, the elongate shaft includes a stiffer proximal segment disposed proximal of the distal end region.

Alternatively or additionally to any of the embodiments above, the pre-shaped curve has a proximal end and wherein the stiffer proximal segment extends distal of the proximal end.

Alternatively or additionally to any of the embodiments above, the pre-shaped curve has a fourth radius of curvature different from the first radius of curvature.

Alternatively or additionally to any of the embodiments above, the fourth radius of curvature is larger than the first radius of curvature.

Alternatively or additionally to any of the embodiments above, the elongate shaft includes a steering wire for bending the distal end region.

Alternatively or additionally to any of the embodiments above, the second direction is opposite the first direction.

A deflectable medical device is disclosed. The deflectable medical device comprises: an elongate shaft having a distal end region; wherein the distal end region defines a first reach distance when bending the distal end region in a first direction prior to having a pre-shaped curve formed therein; and wherein the distal end region defines a second reach distance greater than the first reach distance when bending the distal end region in the first direction after having the pre-shaped curve formed therein.

Alternatively or additionally to any of the embodiments above, the distal end region defines a first radius of curvature when bending the distal end region in the first direction prior to having the pre-shaped curve formed therein and wherein the distal end region defines a second radius of curvature when bending the distal end region in the first direction after having the pre-shaped curve formed therein.

Alternatively or additionally to any of the embodiments above, the first radius of curvature is substantially equal to the second radius of curvature.

Alternatively or additionally to any of the embodiments above, the distal end region defines a third radius of curvature when bending the distal end region in a second direction different from the first direction after having the pre-shaped curve formed therein and wherein the second radius of curvature is larger than the third radius of curvature.

Alternatively or additionally to any of the embodiments above, the elongate shaft includes a stiffer proximal segment disposed proximal of the distal end region.

Alternatively or additionally to any of the embodiments above, the pre-shaped curve has a proximal end and wherein the stiffer proximal segment extends distal of the proximal end.

Alternatively or additionally to any of the embodiments above, the elongate shaft includes a steering wire for bending the distal end region.

A deflectable medical device is disclosed. The deflectable medical device comprises: an elongate shaft having a distal end region; wherein the distal end region defines a first radius of curvature when bending the distal end region in a first direction prior to having a pre-shaped curve formed therein; wherein the distal end region defines a second radius of curvature when bending the distal end region in the first direction after having the pre-shaped curve formed therein; wherein the distal end region defines a third radius of curvature when bending the distal end region in a second direction different from the first direction after having the pre-shaped curve formed therein; wherein the third radius of curvature is smaller than the first radius of curvature, the second radius of curvature, or both the first radius of curvature and the second radius of curvature; wherein the distal end region defines a first reach distance when bending the distal end region in the first direction prior to having the pre-shaped curve formed therein; and wherein the distal end region defines a second reach distance greater than the first reach distance when bending the distal end region in the first direction after having the pre-shaped curve formed therein.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view schematically depicting an example deflectable medical device.

FIG. 2 is a cross-sectional view taken through line 2—2 in FIG. 1.

FIGS. 3-5 illustrate an example deflectable medical device prior to having a pre-shaped curve formed therein.

FIGS. 6-8 illustrate an example deflectable medical device after having a pre-shaped curve formed therein.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

A number of medical interventions may require a clinician to navigate a medical device into the heart. For example, treatment of the left atrial appendage by implanting an occlusive device (e.g., WATCHMAN FLX™ or WATCHMAN FLX™ Pro from Boston Scientific) may require that the device has sufficient reach and/or the ability to navigate with tight deflection/maneuverability. If the intervention results in the clinician performing a pulmonary vein isolation (PVI) or PVI+ (PVI plus adjuvant atrial ablations) maneuvers, which may include septal line, mitral isthmus ablation, and/or the like, even further reach and/or tighter deflections may be desired. In some instances, a clinician may perform a primary intervention (e.g., left atrial appendage occlusion) using a first sheath. In order to perform a secondary intervention (e.g., PVI and/or a PVI+ maneuver), a clinician may choose to remove the first sheath and replace with a second sheath with different properties such as different reach and/or different deflection properties. Disclosed herein are sheaths, for example steerable sheath, that have desirable levels of reach as well as the ability to navigate with tight deflection/maneuverability. Such sheaths may allow a clinician to perform a number of different medical interventions without needing to replace the sheath.

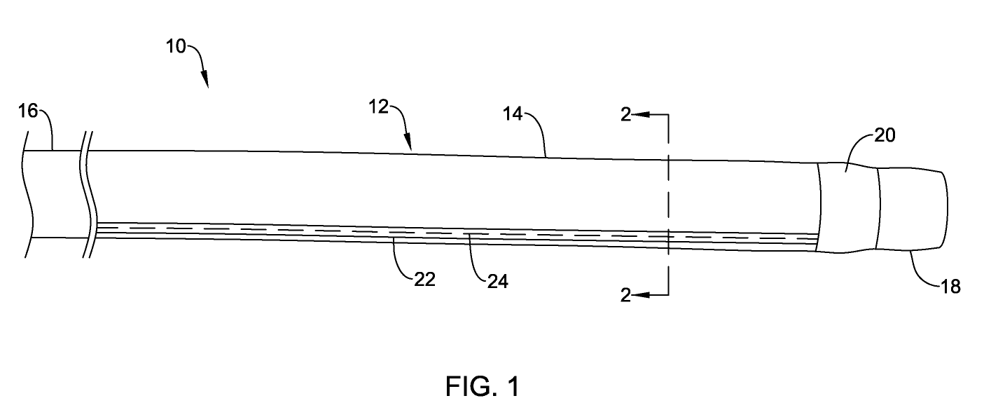

FIG. 1 schematically illustrates an example deflectable medical device 10. The deflectable medical device 10 may include an elongate shaft 12 having a distal end region 14 and a proximal end region 16 disposed proximal of the distal end region 14. The elongate shaft 12 may include a distal tip 18. The elongate shaft 12 may be steerable. As such, the elongate shaft 12 may include a steering member 20. A steering wire 24 may be coupled to the steering member 20. Actuating the steering wire 24 (e.g., pulling the steering wire 24) may cause the distal end region 14 to curve or bend. In FIG. 1, a singular steering wire 24 is depicted enabling unidirectional bending. Alternative elongate shafts are contemplated that include two or more steering wires 24, enabling bidirectional or multidirectional steering (including steering in multiple planes).

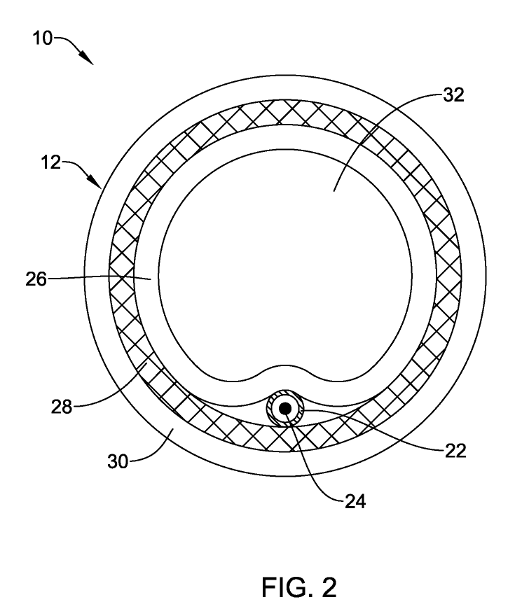

FIG. 2 is a cross-sectional view of the elongate shaft 12. Here the form of the elongate shaft 12, which may vary, can be seen. For example, the elongate shaft 12 may be formed from a plurality of layers such as an inner layer or liner 26, a reinforcing member or layer 28, and an outer layer 30. The inner layer 26 may include a lubricious material such as polytetrafluoroethylene. Other materials including those disclosed herein may be utilized. The reinforcing layer 28 may include a braid, a coil, both a braid and a coil, and/or the like. In some instances, the reinforcing layer 28 may include a slotted tube (e.g., a slotted hypotube, a slotted nickel-titanium alloy tube, etc.). The outer layer 30 may include a polyether block amide. Other materials including those disclosed herein may be utilized. In at least some instances, the outer layer 30 may include a plurality of different sections where more distal sections have an increased flexibility and/or lower durometer relative to more proximal sections. For example, a distal section of the outer layer 30 adjacent to the distal end region 14 of the elongate shaft 12 may include a relatively flexible material and a proximal region of the outer layer 30 (e.g., which may be along the proximal end region 16 of the elongate shaft 12) may include a less flexible material.

A low friction tube 22 may be disposed within the elongate shaft 12. A lumen 32 may be disposed centrally within the elongate shaft 12 for performing primary (e.g., left atrial appendage occlusion) or secondary intervention (e.g., PVI and/or a PVI+ maneuver) or both. The low friction tube 22 may resemble a small/miniature catheter shaft and may be disposed along the inner wall of the elongate shaft 12. Alternatively, the low friction tube 22 may be integrated between the inner layer 26 and the reinforcing layer 28 of the elongate shaft 12. In some instances, the low friction tube 22 may be formed from a single layer of lubricious material or a number of layers. For example, the low friction tube 22 may include an inner lubricious layer (e.g., formed from polytetrafluoroethylene or the like), a reinforcing braid, and an outer layer formed from polyether block amide. Other materials including those disclosed herein may be utilized. As indicated above, the steering wire 24 may be disposed within the low friction tube 22.

The deflectablity (e.g., the ability to navigate with tight deflection/maneuverability) and/or reach of the elongate shaft 12 may be enhanced. Because of this, the deflectable medical device 10 may be capable of being used for a number of different medical interventions, including interventions where multiple procedures are being performed without needing to replace the deflectable medical device 10 between procedures.

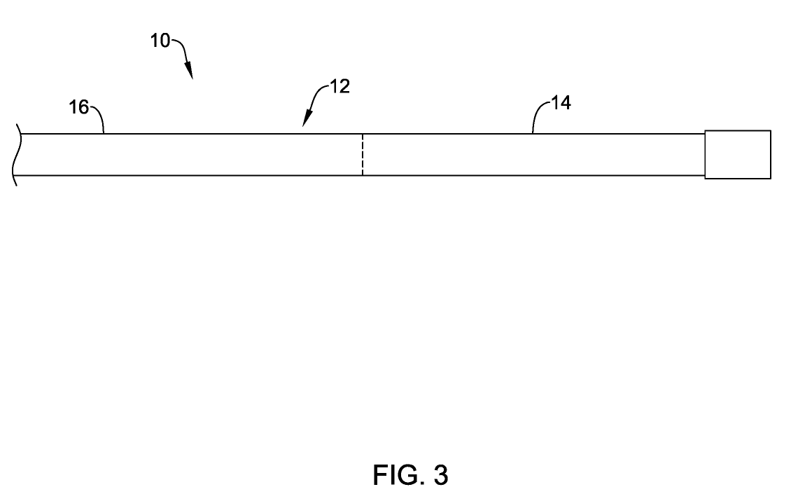

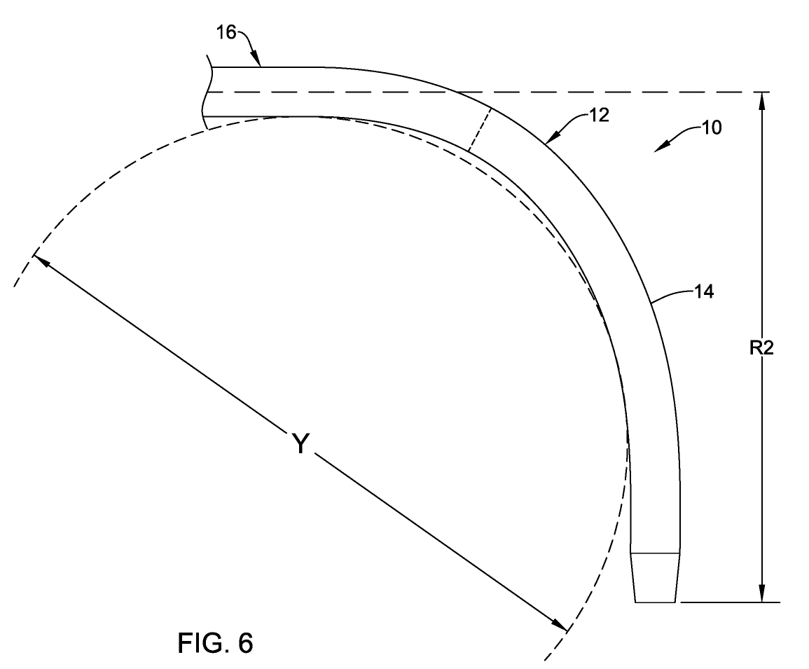

In at least some instances, the deflectability and/or reach of the deflectable medical device 10 may be altered by pre-forming a curve in the elongate shaft 12 and/or by controlling physical properties of the elongate shaft 12. FIGS. 3-5 illustrate the elongate shaft 12 prior to having a pre-shaped curve formed therein. FIGS. 6-8 illustrate the elongate shaft 12 after having the pre-shaped curve formed therein and the corresponding impact on deflectability and/or reach.

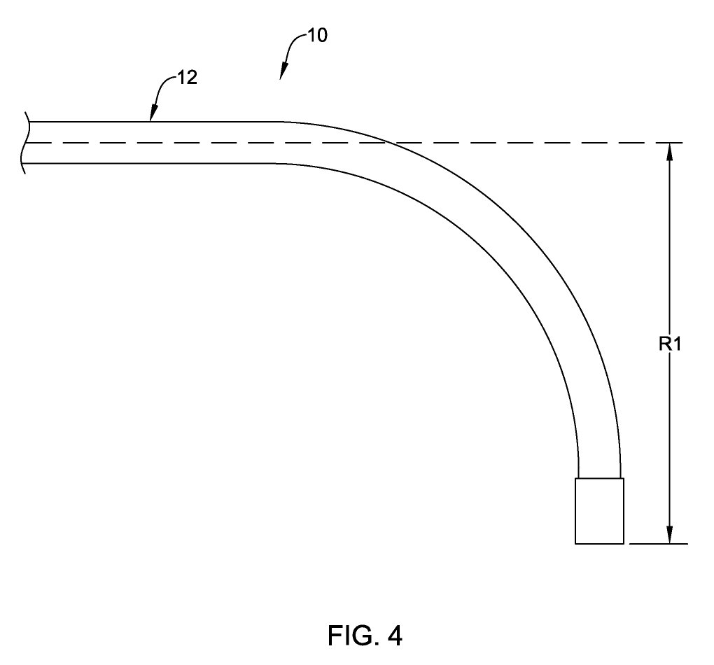

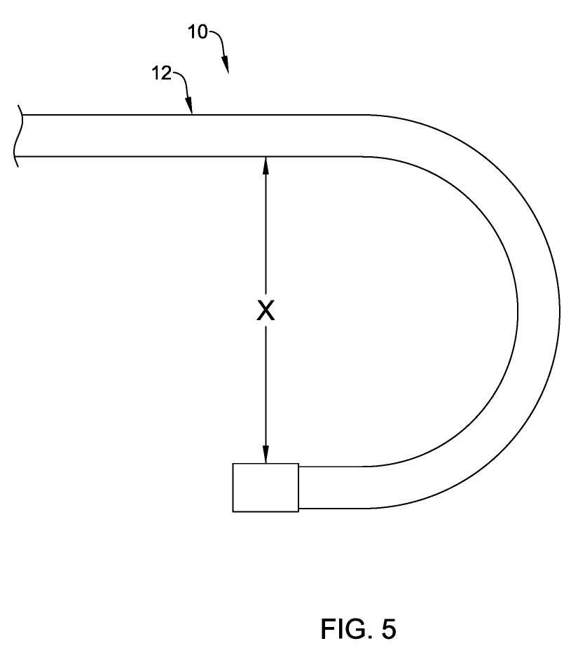

Turning now to FIGS. 3-5, FIG. 3 illustrates the elongate shaft 12 in a non-deflected or “straight” configuration. In FIG. 3, a dashed line is shown that extends across the elongate shaft 12. This line is intended to represent a transition between the proximal end region 16 and the distal end region 14. In at least some instances, this transition may be a position where the outer layer 30 transitions or changes from a less flexible material (e.g., along the proximal end region 16 of the elongate shaft 12) to a more flexible material (e.g., along the distal end region 14 of the elongate shaft 12). This transition may also form or define a transition between a deflection segment (e.g., distal to the dashed line/transition) and a non-deflection segment (e.g., proximal to the dashed line/transition). When the elongate shaft 12 is bent, for example by actuating the steering wire 24, the elongate shaft 12 can be seen to have a reach or reach distance R1 as shown in FIG. 4. FIG. 5 illustrates the elongate shaft 12 bent, for example fully bent in a first direction, so as to define a first radius of curvature, represented in FIG. 5 by depicting the diameter X (noting that the radius of curvature is half the diameter).

As indicated above, it has been found that forming a pre-curve in the elongate shaft 12 can desirably impact the deflectability and/or reach of the elongate shaft 12. FIGS. 6-8 illustrate the elongate shaft 12 after having a pre-shaped curve formed therein. For example, FIG. 6 illustrates a pre-shaped curve having a fourth radius of curvature, represented in FIG. 6 by depicting the diameter Y (noting that the radius of curvature is half the diameter), formed in the elongate shaft 12. FIG. 6 also illustrates the elongate shaft 12 bent or steered so as to define a reach or reach distance R2. The reach distance R2 is greater than the reach distance R1. This may allow the elongate shaft 12 to more efficiently navigate and/or reach different anatomical regions during an intervention.

It can be understood that the distal end region 14 may have or define a first reach distance (e.g., the reach distance R1) when bending the distal end region 14 in a first direction prior to having a pre-shaped curve formed therein. The distal end region 14 may have or define a second reach distance (e.g., the reach distance R2) when bending the distal end region 14 in the first direction after having the pre-shaped curve formed therein. The second reach distance (e.g., the reach distance R2) may be larger than the first reach distance (e.g., the reach distance R1).

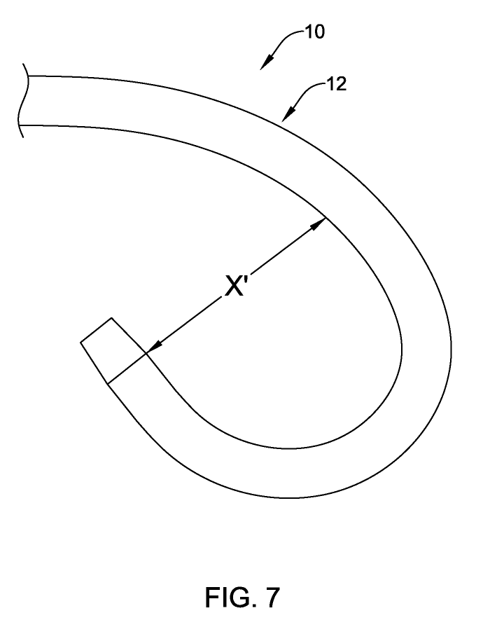

FIG. 7 illustrates the elongate shaft 12 bent, for example fully bent in a first direction, so as to define a second radius of curvature, represented in FIG. 7 by depicting the diameter X’ (noting that the radius of curvature is half the diameter). In some instances, the second radius of curvature may be understood to be the radius of curvature of the pre-shaped curve. Alternatively, the second radius of curvature may be understood to be the radius of curvature of the distal end region 14 when bending the distal end region 14 of the elongate shaft 12 in a first direction after having the pre-shaped curve formed therein. The second radius of curvature may be substantially the same as the first radius of curvature (e.g., the first radius of curvature in the elongate shaft 12 prior to having the pre-shaped curve formed therein), smaller than the first radius of curvature (e.g., about 20% or less smaller, or about 10% or less smaller, or about 5% or less smaller), or larger than the first radius of curvature (e.g., about 20% or less larger or about 10% or less larger, or about 5% or less larger). Because of the pre- shaped curve formed in the elongate shaft 12, the bent/steered arrangement/orientation of the elongate shaft 12 may differ from that of prior to forming the pre-shaped curve. For example, when the elongate shaft 12 is fully bent/steered (e.g., as shown in FIG. 5), the distal tip of the elongate shaft 12 is oriented or otherwise points in a proximal direction (e.g., straight proximally). In other words, the distal tip of the elongate shaft 12 is substantially parallel to the remainder or more proximal portions of the elongate shaft 12. Conversely, after forming the pre-shaped curve in the elongate shaft 12, when the elongate shaft 12 is fully bent/steered (e.g., as shown in FIG. 7), the distal tip of the elongate shaft 12 points proximally as well as laterally toward the remainder or more proximal portions of the elongate shaft 12.

It can be understood that the distal end region 14 of the elongate shaft 12 may define a first radius of curvature (e.g., the first radius of curvature) when bending the distal end region 14, for example in a first direction, prior to having a pre-shaped curve formed therein. The distal end region 14 may define a second radius of curvature (e.g., the second radius of curvature) when bending the distal end region 14 in the first direction after having the pre-shaped curve formed therein. The first radius of curvature may be substantially the same as, larger than, or smaller than the second radius of curvature. For example, the first radius of curvature may be about 8-22 mm, or about 10-20 mm, or about 13-18 mm, or about 13 mm, or about 15 mm, or about 18 mm. The second radius of curvature may be about 10-25 mm, or about 12-22 mm, or about 15-20 mm, or about 15 mm, or about 18 mm, or about 20 mm.

It is noteworthy that the form and/or arrangement of the pre-shaped curve may vary. In at least some instances, the pre-shaped curve may be a singular, constant curve having a constant radius of curvature (e.g., a fourth radius of curvature). The fourth radius of curvature may be the same as or different from (e.g., larger than) the first radius of curvature. For example, the fourth radius of curvature may be about 50-100 mm, or about 55-90 mm, or about 60-85 mm, or about 64-84 mm, or about 64 mm, or about 74 mm, or about 84 mm. In other instances, the pre-shaped curve may have a variable radius of curvature and/or otherwise include regions with different bending radii. This may include pre-shaped curves or curved regions that extend in different planes. In some instances, the pre-shaped curve may be offset from the bending direction of the shaft. For example, the pre-shaped curve may lie within a plane that is offset (e.g., perpendicular) to the plane in the shaft bends (e.g., using the steering wires). Pre-shaped curves are also contemplated where one or more substantially straight sections may be disposed along and disrupt the pre-shaped curve. For example, the pre-shaped curve may include about a 25-40 mm, about a 30-36 mm, or about a 34 mm radius of curvature and a straight section that is about 25-50 mm, or about 30-45 mm, or about 43 mm. In some of these and in other instances, a substantially straight (e.g., non-curved) section may be disposed distally of the pre-shaped curve. It can be appreciated that such a section may even further increase the reach of the elongate shaft 12.

In at least some instances, the pre-shaped curve may include at least a portion of the proximal end region 16 and/or a stiffer portion of the elongate shaft 12. This may include the pre-shaped curve extending across the transition in flexibility (represented in FIG. 6 by a dashed line extending across the elongate shaft 12). For example, as indicated above, the transition may form or define a transition between a deflection segment (e.g., distal to the dashed line/transition) and a non-deflection segment (e.g., proximal to the dashed line/transition). In at least some instances, at least a portion of the non-deflection segment may be disposed along the pre-shaped curve. Stated another way, the pre-shaped curve may be understood to have a proximal end (e.g., where the pre-shaped curve beings) and the non-deflection segment and/or at least a portion of the stiffer proximal segment extends distal of the proximal end.

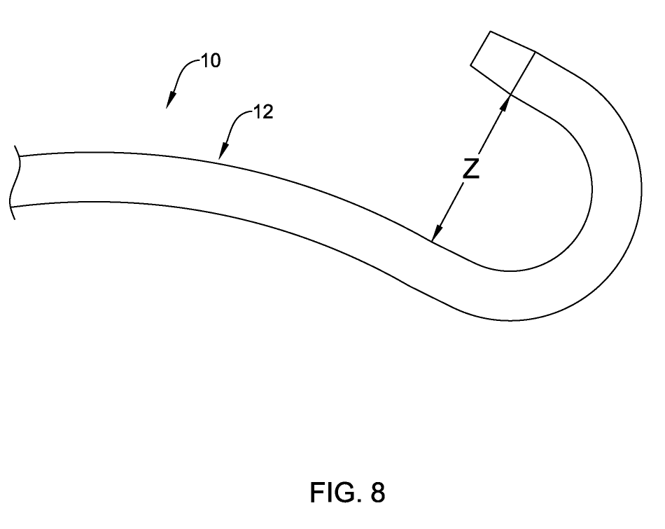

The impact of forming the pre-shaped curve in the elongate shaft 12 (e.g., the distal end region 14) can also impact the deflectability characteristics of the elongate shaft 12 when bending the distal end region 14 in different directions. For example, FIG. 8 illustrates the elongate shaft 12 (e.g., after having the pre-shaped curve formed therein) bent, for example fully bent in a second direction (e.g., opposite the first direction), so as to define a third radius of curvature, represented in FIG. 8 by depicting the diameter Z (noting that the radius of curvature is half the diameter). The third radius of curvature may be different from the first radius of curvature (e.g., the first radius of curvature prior to having the pre-shaped curve formed therein) and/or different from the second radius of curvature (e.g., the second radius of curvature after having the pre-shaped curve formed therein). For example, the third radius of curvature may be smaller than the first radius of curvature (e.g., the first radius of curvature prior to having the pre-shaped curve formed therein) and/or smaller than the second radius of curvature (e.g., the second radius of curvature after having the pre-shaped curve formed therein). In some instances, the third radius of curvature may be about 5-20 mm, or about 6- 18 mm, or about 8-15 mm, or about 8 mm, or about 10 mm, or about 13 mm, or about 15 mm. The ratio of the third radius of curvature to the first radius of curvature (and/or the second radius of curvature) may be about 0.50-0.98, or about 0.55-0.97, or about 0.60-0.95, or about 0.60, or about 0.75, or about 0.80, or about 0.95. In addition, the reach distance of the distal end region 14 when bent in the second direction may also differ (e.g., may be smaller) from other reach distances disclosed herein, which may be utilized when navigating tight anatomies.

The materials that can be used for the various components of the deflectable medical device 10 may include those commonly associated with medical devices. For simplicity purposes, the following discussion makes reference to the elongate shaft 12. However, this is not intended to limit the devices and methods described herein, as the discussion may be applied to other similar tubular members and/or components of tubular members or devices disclosed herein.

The elongate shaft 12 and/or other components of the deflectable medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the deflectable medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the deflectable medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the deflectable medical device 10 to achieve the same result.

In some embodiments, a degree of Magnetic Resonance Imaging (MRI) compatibility is imparted into the deflectable medical device 10. For example, the deflectable medical device 10, or portions thereof, may be made of a material that does not substantially distort the image and create substantial artifacts (e.g., gaps in the image). Certain ferromagnetic materials, for example, may not be suitable because they may create artifacts in an MRI image. The deflectable medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A deflectable medical device, comprising:
an elongate shaft having a distal end region;
wherein the distal end region defines a first radius of curvature when bending the distal end region in a first direction prior to having a pre-shaped curve formed therein;
wherein the distal end region defines a second radius of curvature when bending the distal end region in the first direction after having the pre-shaped curve formed therein;
wherein the distal end region defines a third radius of curvature when bending the distal end region in a second direction different from the first direction after having the pre-shaped curve formed therein; and
wherein the second radius of curvature is larger than the third radius of curvature.

2. The deflectable medical device of claim 1, wherein the first radius of curvature is smaller than the second radius of curvature.

3. The deflectable medical device of claim 1, wherein the first radius of curvature is larger than the second radius of curvature.

4. The deflectable medical device of claim 1, wherein the first radius of curvature is substantially equal to the second radius of curvature.

5. The deflectable medical device of claim 1, wherein the distal end region defines a first reach distance when bending the distal end region in the first direction prior to having the pre-shaped curve formed therein, wherein the distal end region defines a second reach distance when bending the distal end region in the first direction after having the pre-shaped curve formed therein, and wherein the first reach distance is different from the second reach distance.

6. The deflectable medical device of claim 5, wherein the second reach distance is larger than the first reach distance.

7. The deflectable medical device of claim 1, wherein the elongate shaft includes a stiffer proximal segment disposed proximal of the distal end region.

8. The deflectable medical device of claim 7, wherein the pre-shaped curve has a proximal end and wherein the stiffer proximal segment extends distal of the proximal end.

9. The deflectable medical device of claim 1, wherein the pre-shaped curve has a fourth radius of curvature different from the first radius of curvature.

10. The deflectable medical device of claim 9, wherein the fourth radius of curvature is larger than the first radius of curvature.

11. The deflectable medical device of claim 1, wherein the elongate shaft includes a steering wire for bending the distal end region.

12. The deflectable medical device of claim 1, wherein the second direction is opposite the first direction.

13. A deflectable medical device, comprising:
an elongate shaft having a distal end region;
wherein the distal end region defines a first reach distance when bending the distal end region in a first direction prior to having a pre-shaped curve formed therein; and
wherein the distal end region defines a second reach distance greater than the first reach distance when bending the distal end region in the first direction after having the pre-shaped curve formed therein.

14. The deflectable medical device of claim 13, wherein the distal end region defines a first radius of curvature when bending the distal end region in the first direction prior to having the pre-shaped curve formed therein and wherein the distal end region defines a second radius of curvature when bending the distal end region in the first direction after having the pre-shaped curve formed therein.

15. The deflectable medical device of claim 14, wherein the first radius of curvature is substantially equal to the second radius of curvature.

16. The deflectable medical device of claim 14, wherein the distal end region defines a third radius of curvature when bending the distal end region in a second direction different from the first direction after having the pre-shaped curve formed therein and wherein the second radius of curvature is larger than the third radius of curvature.

17. The deflectable medical device of claim 13, wherein the elongate shaft includes a stiffer proximal segment disposed proximal of the distal end region.

18. The deflectable medical device of claim 17, wherein the pre-shaped curve has a proximal end and wherein the stiffer proximal segment extends distal of the proximal end.

19. The deflectable medical device of claim 13, wherein the elongate shaft includes a steering wire for bending the distal end region.

20. A deflectable medical device, comprising:
an elongate shaft having a distal end region;
wherein the distal end region defines a first radius of curvature when bending the distal end region in a first direction prior to having a pre-shaped curve formed therein;
wherein the distal end region defines a second radius of curvature when bending the distal end region in the first direction after having the pre-shaped curve formed therein;
wherein the distal end region defines a third radius of curvature when bending the distal end region in a second direction different from the first direction after having the pre-shaped curve formed therein;
wherein the third radius of curvature is smaller than the first radius of curvature, the second radius of curvature, or both the first radius of curvature and the second radius of curvature;
wherein the distal end region defines a first reach distance when bending the distal end region in the first direction prior to having the pre-shaped curve formed therein; and
wherein the distal end region defines a second reach distance greater than the first reach distance when bending the distal end region in the first direction after having the pre-shaped curve formed therein.
